(19) United States
(12) Patent Application Publication
(10) Pub. No.: US 2026/0035685 A1
(43) Pub. Date: Feb. 5, 2026

(54) METHODS OF RNA PURIFICATION

(71) Applicant: MODERNATX, INC., Cambridge, MA (US)

(72) Inventors: Nagashree Babu, Cambridge, MA (US); Amy E. Rabideau, Cambridge, MA (US); Christopher Ladd Effio, Cambridge, MA (US)

(73) Assignee: MODERNATX, INC., Cambridge, MA (US)

(21) Appl. No.: 18/998,898

(22) PCT Filed: Jul. 27, 2023
(86) PCT No.: PCT/US2023/028816

§ 371 Date: Jan. 27, 2025

Related U.S. Application Data
(60) Provisional application No. 63/393,197, filed on Jul. 28, 2022.

(51) Int. Cl. C12N 15/10

(52) U.S. Cl. CPC C12N 15/101

(57) ABSTRACT

Provided herein, in some embodiments, are methods of purifying low-salt RNA compositions using a flow-through column comprising hydrophobic interaction chromatography (HIC) resin having high hydrophobicity.

### RELATED APPLICATION

This application claims the benefit under 35 U.S.C. § 119 (c) of U.S. provisional application No. 63/393,197, filed Jul. 28, 2022, which is incorporated by reference herein in its entirety.

### BACKGROUND

In vitro transcription (IVT) uses bacteriophage DNA-dependent ribonucleic acid (RNA) polymerases (e.g., SP6, T3 and T7) to synthesize template-directed messenger RNA (mRNA) transcripts. Problems in an IVT reaction can result in complete failure (e.g., no transcript generated) or in transcripts that are the incorrect size (e.g., shorter or longer than expected), for example. Specific problems associated with IVT reactions include, for example, abortive (truncated) transcripts, run-on transcripts, poly-A tail variants/3′ heterogeneity (including low percent poly-A tailed mRNA), mutated transcripts, and/or double-stranded contaminants produced during the reactions. One mechanism to counteract these problems resulting from IVT reactions is to purify the mRNA products after the reaction is complete.

### SUMMARY

The present disclosure provides, in some embodiments, methods of isolating a high yield of highly pure ribonucleic acid (RNA), such as messenger RNA (mRNA), for example, from an in vitro transcription (IVT) reaction. Previous mRNA purifications have centered on the use of ambient oligo-dT alone or in combination with reverse-phase HPLC. However, these purification methods provide low percent tailed mRNA purity (ambient oligo-dT alone), exhibit low clearance of proteins (e.g., enzymes used during an IVT reaction), and/or are cost-prohibitive at large scale (ambient oligo-dT in combination with reverse-phase HPLC). As such, new RNA purification methods are needed. Surprisingly, studies herein show that methods of purifying

RNA using a flow-through column comprising hydrophobic interaction chromatography (HIC) resin, wherein the mixture comprising RNA is a low-salt mixture, and/or wherein the HIC resin has high hydrophobicity, produce highly purified RNA (e.g., RNA produced from an IVT reaction) with very low quantities of residual protein. In some embodiments, the inventors have shown that methods of purifying RNA using a combination of flow-through hydrophobic interaction chromatography (HIC) and denaturing oligo-dT resin (e.g., denaturing oligo-dT, e.g., under low-salt conditions) produce highly purified RNA (e.g., RNA produced from an IVT reaction) with very low quantities of residual protein. In some embodiments, the methods described herein produce compositions comprising purified RNA without any detectable residual protein. These methods described herein may also demonstrate higher efficiency than previously described methods. For example, a combination of flow-through HIC and denaturing oligo-dT has been shown to have a 15.8-fold improvement in chromatography productivity (g/L/h) relative to previously described processes. This increase of efficiency means that more RNA can be produced and purified in the same amount of time, leading to decreased financial and material costs.

Thus, aspects of the present disclosure provide methods comprising applying a mixture comprising messenger ribonucleic acid (mRNA) produced by an in vitro transcription reaction to a flow-through column comprising hydrophobic interaction chromatography (HIC) resin, wherein the mixture comprising mRNA is a low-salt mixture, and wherein the HIC resin has high hydrophobicity. In addition, in some aspects, the present disclosure provides methods comprising applying a mixture comprising messenger ribonucleic acid (mRNA) produced by an in vitro transcription reaction and residual protein to a flow-through column comprising hydrophobic interaction chromatography (HIC) resin under conditions that do not allow for the mRNA to substantially bind to the HIC resin but do allow for the residual protein to bind to the HIC resin.

In some embodiments, conditions that do not allow for the mRNA to substantially bind to the HIC resin but do allow for the residual protein to bind to the HIC resin comprise low-salt conditions, optionally wherein the mixture comprising mRNA is a low-salt mixture. In some embodiments, a method further comprises desalting the mixture prior to applying the mixture to the flow-through column. In some embodiments, the low-salt mixture comprises a salt concentration of less than 20 mM. In some embodiments, the low-salt mixture comprises a salt concentration of 0-500 mM, optionally 0-350 mM.

In some embodiments, the HIC resin is equilibrated prior to the applying step using a buffer solution comprising 0-100 mM salt concentration, optionally 25 mM. In some embodiments, following the applying step, the HIC resin is washed with a buffer solution comprising 0-100 mM salt concentration, optionally 25 mM. In some embodiments, the salt comprises an alkali metal cation, optionally wherein the alkali metal is sodium or potassium. In some embodiments, the salt comprises an alkaline earth metal, optionally wherein the alkaline earth metal is magnesium. In some embodiments, the mixture or buffer solution further comprises a counterion, optionally wherein the counterion is chloride, phosphate, or sulfate. In some embodiments, the salt comprises an anti-chaotropic salt, optionally wherein the anti-chaotropic salt is ammonium sulfate.

In some embodiments, the mRNA does not substantially bind to the hydrophobic interaction resin and/or residual protein binds to the HIC resin. In some embodiments, the HIC resin comprises a cross-linked poly (styrene-divinylbenzene) matrix with an aromatic hydrophobic benzyl ligand and an average particle size of 50 μm. In some embodiments, the HIC resin comprises a hydrophobic moiety selected from butyl, t-butyl, phenyl, ether, amide, or propyl groups.

In some embodiments, the mRNA being applied to the HIC resin comprises at least 90% poly-A tailed mRNA. In some embodiments, a method further comprises isolating the mRNA from the flow-through column. In some embodiments, a method further comprises applying the RNA to a tangential flow filtration step following isolating the mRNA from the flow-through column.

### BRIEF DESCRIPTION OF THE DRAWINGS

FIG. 1 provides graphs showing the residual protein (% w/w, determined by an ELISA assay) in compositions comprising RNA produced by IVT following purification using a flow-through column comprising one of several tested hydrophobic interaction chromatography (HIC) resins (Benzyl Ultra, Benzyl, and Phenyl High Sub).

FIG. 2 is a graph showing the ability of ambient oligo-dT to remove residual protein from an IVT reaction.

FIG. 3 is a graph showing the ability of an exemplary method of the disclosure that utilizes flow-through HIC to remove residual protein from an IVT reaction.

### DETAILED DESCRIPTION

In vitro transcription (IVT) reactions present a powerful platform for the production of RNA (e.g., mRNA). Nonetheless, in addition to producing the desired RNA product(s), IVT reactions also generate significant levels of impurities (including the enzymes and protein used during the reaction). In part because of those impurities, purification of the desired RNA product(s) has proved to be a challenge. Provided herein, in some embodiments, are methods for efficient, cost-effective removal of RNA impurities from large-scale IVT reactions. These methods utilize flow-through hydrophobic interaction chromatography (HIC) resin to purify RNA from low-salt mixtures (e.g., low-salt mixtures comprising mRNA produced using an IVT reaction and impurities including residual protein from the IVT reaction), wherein the HIC resin has high hydrophobicity. In some embodiments, methods utilize flow-through hydrophobic interaction chromatography (HIC) resin under conditions that do not allow RNA to substantially bind to the HIC resin but do allow protein (e.g., residual protein from an IVT reaction and/or downstream processing) to bind to the HIC resin. In some embodiments, the method further utilizes a purification step involving denaturing oligo-dT resin. In some embodiments, the method comprises applying denaturing conditions to a mixture comprising ribonucleic acid (RNA) to produce a composition comprising denatured RNA; binding the denatured RNA to an oligo-dT resin; eluting RNA from the oligo-dT resin; applying the eluted RNA to a flow-through column comprising hydrophobic interaction chromatography (HIC) resin; and isolating the RNA. Thus, as described herein, the present disclosure provides methods of purifying mixtures comprising RNA (e.g., mRNA produced by an IVT reaction).

In some embodiments, a purified mixture comprising RNA (e.g., following flow-through HIC) does not comprise detectable levels (e.g., detectable quantities) of residual protein. e.g. In some embodiments, a purified mixture comprising RNA (e.g., following flow-through HIC) comprises less than 5% w: w (weight protein relative to total weight of mixture), less than 4% w: w, less than 3% w: w, less than 2% w: w, less than 1% w: w, less than 0.5% w: w, less than 0.3% w: w, less than 0.1% w: w, less than 0.05% w: w, less than 0.025% w: w, less than 0.01%, less than 0.005% w: w, or less than 0.001% residual protein. In some embodiments, a purified mixture comprising RNA (e.g., following flow-through HIC) comprises 0.01% to 5% w: w (weight protein relative to total weight of mixture), 0.01% to 4% w: w, 0.05% to 3% w: w. 0.05% to 2% w: w. 0.05% to 1% w: w. 0.05% to 0.5% w: w, 0.05% to 0.3% w: w. 0.01% to 0.1% w: w, 0.01% to 0.05% w: w. 0.01% to 0.025% w: w. 0.01% to 0.02% w: w. 0.001% to 0.01%, or 0.001% to 0.05% w: w residual protein.

### In Vitro Transcription (IVT) Reaction Mixture

In some embodiments, the mixture or RNA composition to be purified or isolated using the methods described herein is produced by an in vitro transcription (IVT) reaction. In some embodiments, IVT reactions require a linear DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. IVT reactions may be performed by incubating a DNA template with an RNA polymerase and nucleoside triphosphates, including GTP, ATP, CTP, and UTP (or nucleotide analogs) in a transcription buffer. In some embodiments, an RNA polymerase for use in an IVT reaction is as described in WO2019/036682 or WO2020/172239. In some embodiments, an IVT reaction is a bolus fed-batch IVT reaction, a continuous fed-batch IVT reaction, or a batch IVT reaction. In some embodiments, an RNA transcript having a 5′ cap structure is produced from this reaction.

A DNA template may include a polynucleotide encoding a polypeptide of interest (e.g., an antigenic polypeptide). A DNA template, in some embodiments, includes an RNA polymerase promoter (e.g., a T7 RNA polymerase promoter) that is operably linked to a polynucleotide encoding a polypeptide of interest. In some embodiments, a DNA template can be transcribed by an RNA polymerase. A DNA template may also include a nucleotide sequence encoding a poly-Adenylation (poly-A) tail at the 3′ end of the polynucleotide encoding a polypeptide of interest.

An RNA, in some embodiments, is the product of an IVT reaction. An RNA, in some embodiments, is a messenger RNA (mRNA) that includes a nucleotide sequence encoding a polypeptide of interest linked to a poly-A tail.

A “poly-A tail” is a region of RNA that contains multiple, consecutive adenosine monophosphates that is downstream, from the region encoding a polypeptide of interest (e.g., directly downstream of the 3′ untranslated region). A poly-A tail may contain 10 to 300 adenosine monophosphates. For example, a poly-A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly-A tail contains 50 to 250 adenosine monophosphates. In some embodiments, a poly-A tail may contain fewer than 10 adenosine monophosphates (e.g., 2, 3, 4, 5, 6, 7, 8, or 9).

In some embodiments, percent tailed RNA (the percent of RNA transcripts comprising a poly-A tail) is greater than 50%, greater than 60%, greater than 70%, greater than 80%, greater than 90%, greater than 95% following an IVT reaction. As used herein, percent tailed RNA generally refers to the relative abundance of transcribed RNA product that contains a 3′ poly-A tail. In some embodiments, percent tailed RNA (the percent of transcribed RNA product comprising a 3′ poly-A tail) is greater than 20%, 25%, 30%, 35%, 40%, 45%, 50%, 55%, 60%, 65%, 70%, 75%, 80%, or 85%. In some embodiments, percent tailed RNA is greater than greater than 90%, 95%, 97%, or 99%. In some embodiments, percent tailed RNA (the percent of transcribed RNA product comprising a 3′ poly-A tail) is 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99%.

In some embodiments, the RNA is not chemically modified and comprises the standard ribonucleotides consisting of adenosine, guanosine, cytosine and uridine. In some embodiments, nucleotides and nucleosides of the present disclosure comprise standard nucleoside residues (e.g. A, G, C, or U). In some embodiments, nucleotides and nucleosides of the present disclosure comprise standard deoxyribonucleosides such as those present in DNA (e.g. dA, dG, dC, or dT).

In some embodiments, the RNA is a modified mRNA (mmRNA) and includes at least one modified nucleotide. In some embodiments, the terms “modification” and “modified” refers to modification with respect to adenosine (A), guanosine (G), uridine (U), thymidine (T) or cytidine (C) ribonucleosides or deoxyribonucleosides in at least one of their nucleobase positions, pattern, percent or population. The RNA may comprise modifications that are naturally-occurring, non-naturally-occurring or the RNA may comprise a combination of naturally-occurring and non-naturally-occurring modifications. The RNA may include any useful modification, for example, of a sugar, a nucleobase, or an internucleoside linkage (e.g., to a linking phosphate, to a phosphodiester linkage or to the phosphodiester backbone).

The RNA, in some embodiments, comprises modified nucleosides and/or nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. In some embodiments, modified nucleobases in RNA are selected from the group consisting of pseudouridine (w), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, an RNA includes a combination of at least two (e.g., 2, 3, 4 or more) of the aforementioned modified nucleobases.

In some embodiments, modified nucleobases in RNA are selected from the group consisting of 1-methyl-pseudouridine (mlv), 5-methoxy-uridine (mo5U), 5-methyl-cytidine (m5C), pseudouridine (ψ), α-thio-guanosine and α-thio-adenosine. In some embodiments, an RNA includes a combination of at least two (e.g., 2, 3, 4 or more) of the aforementioned modified nucleobases.

In some embodiments, an RNA comprises pseudouridine (ψ) and 5-methyl-cytidine (m5C). In some embodiments, an RNA comprises 1-methyl-pseudouridine (m1ψ). In some embodiments, an RNA comprises 1-methyl-pseudouridine (m1ψ) and 5-methyl-cytidine (m5C). In some embodiments, an RNA comprises 2-thiouridine (s2U). In some embodiments, an RNA comprises 2-thiouridine and 5-methyl-cytidine (m5C). In some embodiments, an RNA comprises methoxy-uridine (mo5U). In some embodiments, an RNA comprises 5-methoxy-uridine (mo5U) and 5-methyl-cytidine (m5C). In some embodiments, an RNA comprises 2′-O-methyl uridine. In some embodiments an RNA comprises 2′-O-methyl uridine and 5-methyl-cytidine (m5C). In some embodiments, an RNA comprises N6-methyl-adenosine (m6A). In some embodiments, an RNA comprises N6-methyl-adenosine (m6A) and 5-methyl-cytidine (m5C).

The RNA may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G. U, or C.

In some embodiments, the RNA comprises a cap analog. An RNA cap analog generally enhances mRNA stability and translation efficiency. Traditional cap analogs include GpppG, m7GpppG, and m2,2,7GpppG. In some embodiments, an RNA cap analog of the present disclosure is a dinucleotide cap, a trinucleotide cap, or a tetranucleotide cap. In some embodiments, the cap analog is a trinucleotide cap.

In some embodiments, the trinucleotide cap comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises a sequence selected from the following sequences: m7GpppApA, m7GpppApC, m7GpppApG, m7GpppApU, m7GpppCpA, m7GpppCpC, m7GpppCpG, m7GpppCpU, m7GpppGpA, m7GpppGpC, m7GpppGpG, m7GpppGpU, m7GpppUpA, m7GpppUpC, m7GpppUpG, and m7GpppUpU.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

It should be understood that a cap analog, as provided herein, may include any of the cap analogs described in international publication WO 2017/066797, published on 20 Apr. 2017, incorporated by reference herein in its entirety.

As used herein, percent capped RNA generally refers to the relative abundance of transcribed RNA product that contains an incorporated cap analog at its 5′ terminus. In some embodiments, a cap analog is an RNA cap analog. In some embodiments, an RNA cap analog is a dinucleotide, trinucleotide, or tetranucleotide. In some embodiments, percent capped RNA (the percent of transcribed RNA product comprising a 5′ cap analog) is greater than 20%, 25%, 30%, 35%, 40%, 45%, 50%, 55%, 60%, 65%, 70%, 75%, 80%, or 85%. In some embodiments, percent capped RNA is greater than greater than 90%, 95%, 97%, or 99%. In some embodiments, percent capped RNA is between 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99%.

The RNA may be any size or length. In some embodiments, the RNA is 50-250, 200-500, 400-5000, 400-4000, 400-3000, 400-2000, 400-1000, 500-5000, 500-1500, 750-2000, 1000-1500, 1250-2000, 1500-2000, 1750-2500, 2000-3000, 2500-3500, 3000-4000, 3500-4500, or 4000-5000 nucleotides in length.

### Flow-Through Hydrophobic Interaction Chromatography (HIC)

The methods of described herein generally involve the use of a flow-through column comprising hydrophobic interaction chromatography (HIC) resin. In some embodiments, the methods of purifying RNA (e.g., RNA produced by an IVT reaction) comprise applying a mixture comprising RNA produced by an IVT reaction to a flow-through column comprising hydrophobic interaction chromatography (HIC) resin, wherein the mixture comprising RNA is a low-salt mixture, and wherein the HIC resin has high hydrophobicity. In the methods described herein, the RNA generally does not bind to the HIC resin. In the methods described herein, proteins such as residual protein from an IVT reaction, does bind to the HIC resin.

As described herein, HIC resin generally refers to a chromatographic resin or material that functions in use of purification of RNA, separation of RNA from residual protein, and/or isolation of RNA from an impure mixture (e.g., a mixture comprising RNA produced by an IVT reaction) based on hydrophobic interactions between the resin and protein impurities present in the mixture (e.g., residual proteins, e.g., enzymes, from an IVT reaction). In some embodiments, HIC resins comprise hydrophobic moieties that interact with biomolecules such as proteins using hydrophobic interactions. In some embodiments, the hydrophobic moieties are bonded with, attached to, or connected to silica or a related material. In some embodiments, the hydrophobic moiety is selected from alkyl, aryl, butyl, t-butyl, phenyl, ether, amide, or propyl groups. In some embodiments, a HIC resin is a HiTrap Butyl HP resin, CaptoPhenyl resin, Phenyl Sepharose™ 6 resin, Phenyl Sepharose™ High Performance resin, Octyl Sepharose™ High Performance resin, Fractogel™ EMD Propyl resin, Fractogel™ EMD Phenyl resin, Macro-Prep™Methyl resin, HiScreen Butyl FF. HiScreen Octyl FF, or Tosoh Hexyl. In some embodiments, a HIC resin comprises a cross-linked poly (styrene-divinylbenzene) matrix with an aromatic hydrophobic benzyl ligand. In some embodiments, a HIC resin has an average particle size of 5-500 μm, 50-250 μm, 5-100 μm, 10-100 μm, 20-100 μm, 25-75 μm, or 30-60 μm. In some embodiments, a HIC resin has an average particle size of 10 μm, 20 μm, 30 μm, 40 μm, 50 μm, 60 μm, 70 μm, 80 μm, 90 μm, or 100 μm. In some embodiments, the degree of hydrophobicity is defined based on the binding affinity of the HIC resin for a hydrophobic protein. In some embodiments, a HIC resin is a high hydrophobicity HIC resin. In some embodiments, a high hydrophobicity HIC resin comprises a benzyl ligand (e.g., an aromatic hydrophobic benzyl ligand). In some embodiments, a high hydrophobicity HIC resin is a Poros Benzyl resin. Poros Benzyl Ultra resin, or Poros R150 resin. In some embodiments, a high hydrophobicity HIC resin comprises a phenyl ligand or a butyl ligand. In some embodiments, a high hydrophobicity HIC resin is as described in “Increasing productivity in hydrophobic interaction chromatography (HIC) using Capto™ resins.” Cytiva, accessed 27 Jul. 2022. In some embodiments, a high hydrophobicity HIC resin is as described in “POROS™ Hydrophobic InteractionChromatography (HIC) Resins,” Thermo Scientific, accessed 27 Jul. 2022.

The inventors have found that the use of HIC resins are effective in purifying RNA from a mixture comprising RNA produced by an IVT reaction, in part because HIC resin is effective in binding to residual protein in the IVT reaction. The residual protein binds to the HIC resin in select conditions (e.g., low-salt conditions), but the RNA does not. Thus, in a flow-through column comprising HIC resin, the residual protein can bind to the HIC resin under low-salt buffer conditions while the RNA flows through the column (and is purified from the residual protein). In some embodiments, methods utilize flow-through hydrophobic interaction chromatography (HIC) resin under conditions (e.g., low-salt conditions) that do not allow RNA to substantially bind to the HIC resin but do allow for protein (e.g., residual protein from an IVT reaction and/or downstream processing) to bind to the HIC resin. In some embodiments, RNA does not “substantially bind” to the HIC resin if less than 5%, less than 4%, less than 3%, less than 2%, or less than 1% of the total RNA present in a mixture comprising RNA binds to the HIC resin.

In some embodiments, a low-salt buffer condition comprises sodium, potassium, magnesium, manganese, calcium, sulfate, phosphate, and/or chloride salts. In some embodiments, a low-salt buffer condition comprises sodium chloride, sodium phosphate, sodium sulfate, potassium chloride, potassium phosphate, potassium sulfate, magnesium chloride, magnesium phosphate, magnesium sulfate, calcium chloride, calcium phosphate, and/or calcium sulfate. In some embodiments, a low-salt buffer condition comprises a total salt concentration of less than 500 mM, less than 400 mM, less than 300 mM, less than 200 mM, less than 100 mM, less than 50 mM, less than 20 mM, less than 15 mM, less than 10 mM, less than 5 mM, or less than 1 mM. In some embodiments, a low-salt buffer condition comprises a salt concentration of 100-500 mM, 200-500 mM, 100-250 mM. 100-150 mM. 50-150 mM, 50-100 mM. 25-75 mM, 20-100 mM, 1-20 mM, 1-15 mM, 1-10 mM, 1-5 mM, 5-20 mM, 5-10 mM, 10-20 mM, 10-15 mM or 15-20 mM. In some embodiments, a low-salt buffer condition results in a conductivity of less than 2.5 mS/cm, less than 2 mS/cm, less than 1.5 mS/cm, less than 1 mS/cm, or less than 0.5 mS/cm. In some embodiments, a low-salt buffer condition comprises a conductivity of 0.1-2.5 mS/cm. 0.1-2 mS/cm, 0.5-2 mS/cm. 0.5-1 mS/cm. 1-2 mS/cm, 1-1.5 mS/cm, or 1-1.25 mS/cm.

In some embodiments, the HIC resin is equilibrated with a low-salt buffer condition prior to flowing the mixture comprising RNA through the column comprising the HIC resin. Thus, in some embodiments, the HIC resin is equilibrated with a buffer comprising 100-500 mM, 200-500 mM, 100-250 mM. 100-150 mM, 50-150 mM, 50-100 mM, 25-75 mM, 20-100 mM, 1-20 mM, 1-15 mM, 1-10 mM, 1-5 mM, 5-20 mM, 5-10 mM, 10-20 mM, 10-15 mM or 15-20 mM salt concentration. In some embodiments, the HIC resin is equilibrated with a buffer comprising 0-100 mM salt concentration. In some embodiments, the HIC resin is equilibrated with a buffer comprising 0, 5, 10, 15, 20, 25, 30, 40, 50, 60, 70, 80, 90, or 100 mM salt concentration. In some embodiments, a flow-through column comprising HIC resin is equilibrated by flowing an excess of the low-salt buffer through the column. An excess of low-salt buffer may be, in some embodiments, 2-10 column volumes of buffer.

In some embodiments, an RNA mixture (e.g., a mixture comprising RNA produced by an IVT reaction) comprises a low-salt buffer condition when said mixture is applied to a flow-through column comprising a HIC resin (e.g., a HIC resin that has been equilibrated with a low-salt buffer condition). Thus, in some embodiments, an RNA mixture (e.g., a mixture comprising RNA produced by an IVT reaction) comprises a buffer comprising 100-500 mM, 200-500 mM, 100-250 mM, 100-150 mM, 50-150 mM, 50-100 mM, 25-75 mM. 20-100 mM, 1-20 mM, 1-15 mM. 1-10 mM, 1-5 mM, 5-20 mM, 5-10 mM, 10-20 mM, 10-15 mM or 15-20 mM salt concentration. In some embodiments, an RNA mixture (e.g., a mixture comprising RNA produced by an IVT reaction) comprises a buffer comprising 0-100 mM salt concentration. In some embodiments, an RNA mixture (e.g., a mixture comprising RNA produced by an IVT reaction) comprises a buffer comprising 0, 5, 10, 15, 20, 25, 30, 40, 50, 60, 70, 80, 90, or 100 mM salt concentration.

In some embodiments, the same low-salt buffer is used for equilibration of a flow-through column comprising HIC resin and in the mixture comprising RNA. For example, in some embodiments, a column is equilibrated with a low-salt buffer comprising 25 mM NaCl and a mixture comprising RNA produced by an IVT reaction comprises a buffer comprising 25 mM NaCl.

In some embodiments, following application of an RNA mixture to a flow-through column comprising HIC resin, the HIC resin is washed with a low-salt buffer condition. In some embodiments, the washing step is utilized to elute or isolate all RNA from the column, but not remove any bound protein from the HIC resin. Thus, in some embodiments, the HIC resin is washed with a buffer that does not disrupt the binding interactions between the HIC resin and the bound proteins. In some embodiments, the HIC resin is washed with a low-salt buffer comprising 100-500 mM, 200-500 mM, 100-250 mM, 100-150 mM, 50-150 mM, 50-100 mM, 25-75 mM, 20-100 mM, 1-20 mM, 1-15 mM, 1-10 mM, 1-5 mM, 5-20 mM, 5-10 mM, 10-20 mM, 10-15 mM or 15-20 mM salt concentration. In some embodiments, the HIC resin is washed with a buffer comprising 0-100 mM salt concentration. In some embodiments, the HIC resin is washed with a buffer comprising 0, 5, 10, 15, 20, 25, 30, 40, 50, 60, 70, 80, 90, or 100 mM salt concentration. In some embodiments, the RNA flows through the HIC resin during the washing step. In some embodiments, proteins such as residual protein from an IVT reaction remains bound to the HIC resin during the washing step.

A low-salt buffer may comprise an alkali metal cation. In some embodiments, an alkali metal is sodium or potassium. In some embodiments, a low-salt buffer comprises an alkaline earth metal. In some embodiments, an alkaline earth metal is magnesium or manganese. In some embodiments, a low-salt buffer comprises a counterion (e.g., chloride, phosphate, or sulfate). In some embodiments, a low-salt buffer comprises sodium chloride, sodium phosphate, sodium sulfate, potassium chloride, potassium phosphate, potassium sulfate, magnesium chloride, magnesium phosphate, or magnesium sulfate. In some embodiments, a low-salt buffer comprises an anti-chaotropic salt (e.g., ammonium sulfate, carbohydrates such as glucose or trehalose). In some embodiments, an anti-chaotropic salt is a salt or agent that causes water molecules to favorably interact and stabilizes intramolecular interactions in biomolecules such as proteins.

In some embodiments, a buffer solution (e.g., a low-salt buffer condition) may further comprise a buffering agent (e.g., to maintain a consistent pH, e.g., a physiological pH). In some embodiments, a buffer solution (e.g., a low-salt buffer condition) comprises a pH of about 6, about 6.5, about 6.6, about 6.7, about 6.8, about 6.9, about 7, about 7.1, about 7.2, about 7.3, about 7.4, about 7.5, or about 8. In some embodiments, a buffering agent comprises ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis [2-hydroxyethyl]amino)-2-hydroxypropanesulfonic acid (DIPSO), N-(2-hydroxyethyl) piperazine-N′-(2-hydroxypropanesulfonic acid) (HEPPSO), 4-(2-hydroxyethyl)-1-piperazine propanesulfonic acid (EPPS), N-[tris (hydroxymethyl)-methyl]glycine (Tricine), N,N-bis(2-hydroxyethyl) glycine (Bicine), [(2-hydroxy-1,1-bis (hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris (hydroxymethyl) aminomethane (Tris), or bis [2-hydroxyethyl]iminotris-[hydroxymethyl] methane (Bis-Tris).

In some embodiments, a flow-through column comprising HIC resin is a gravity column or a column to which external pressure may be applied. In some embodiments, a column comprises HIC resin that is attached (e.g., covalently attached) to the sides of the column. In other embodiments, a column is packed with HIC resin that is not attached to the sides of the column. A column may be any reasonable size (e.g., comprising a total column volume of 100-1000 mL, 1-500 L, or more). A column may be a research size or a production-scale size.

In some embodiments, the absorbance of the RNA is monitored using ultraviolet detection before and after applying the RNA to the flow-through column comprising HIC resin. In some embodiments, monitoring the RNA using ultraviolet detection allows for determination of the primary structure and/or the secondary structure of the RNA. In some embodiments, monitoring of RNA using UV detection can be performed using an in-line detector. In some embodiments, monitoring of RNA involves taking a measurement of a sample of RNA before applying the RNA to the column and then a second measurement of a second sample of RNA after the RNA has flowed through the column.

In some embodiments, enzymes are incubated with the sample comprising RNA before application of the sample to the column. In some embodiments T4 ligase, pyrophosphatase and/or endonuclease (e.g., DNase I) are incubated with the sample comprising RNA before application of the sample to the column. In some embodiments, this enzymatic incubation prior to application of the sample to the column is performed to degrade non-RNA biomolecules such as residual proteins from an IVT reaction and/or DNA molecules. In some embodiments, following incubation of the sample with enzymes (e.g., T4 ligase, pyrophosphatase and/or endonuclease), the sample is applied to a desalting column. In some embodiments, following incubation of the sample with enzymes (e.g., T4 ligase, pyrophosphatase and/or endonuclease), the sample is applied to a tangential flow filtration step.

### Desalting Mixtures Comprising RNA

In some embodiments, mixtures comprising RNA are desalted in order to produce low-salt RNA compositions (e.g., having less than 20 mM total salt concentration). In some embodiments, a mixture comprising RNA (e.g., a mixture produced by an IVT reaction) is desalted prior to denaturation of the RNA and/or in-line mixing with a high-salt buffer. In some embodiments, a mixture comprising RNA (e.g., a mixture produced by an IVT reaction) is desalted prior to application of the mixture to a flow-through column comprising HIC resin.

In some embodiments, a low-salt RNA composition comprises sodium, potassium, magnesium, manganese, calcium, sulfate, phosphate, and/or chloride salts. In some embodiments, a low-salt RNA composition comprises sodium chloride, sodium phosphate, sodium sulfate, potassium chloride, potassium phosphate, potassium sulfate, magnesium chloride, magnesium phosphate, magnesium sulfate, calcium chloride, calcium phosphate, and/or calcium sulfate. In some embodiments, a low-salt RNA composition comprises a total salt concentration of less than 20 mM, less than 15 mM, less than 10 mM, less than 5 mM, or less than 1 mM. In some embodiments, a low-salt RNA composition comprises a salt concentration of 1-20 mM, 1-15 mM, 1-10 mM, 1-5 mM, 5-20 mM, 5-10 mM, 10-20 mM, 10-15 mM or 15-20 mM. In some embodiments, a low-salt RNA composition results in a conductivity of less than 2.5 mS/cm, less than 2 mS/cm, less than 1.5 mS/cm, less than 1 mS/cm, or less than 0.5 mS/cm. In some embodiments, a low-salt RNA composition comprises a conductivity of 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

In some embodiments, desalting a mixture comprising RNA is accomplished using tangential flow filtration (TFF) of the mixture into a low-salt solution or water, dilution of the mixture with a low-salt solution or water, or ambient oligo-dT (e.g., under native, non-denaturing RNA conditions).

### Denatured RNA

In some embodiments, an RNA composition (e.g., a low-salt RNA composition) is denatured. In some embodiments, a low-salt RNA composition is denatured prior to (e.g., immediately prior to) in-line mixing with a high-salt buffer and subsequent binding of the denatured RNA to an oligo-dT resin.

RNA may be denatured using any method. In some embodiments, RNA is denatured by heating the low-salt RNA composition to 60° C. to 90° C., 60° C. to 80° C., 60° C. to 70° C., 65° C. to 85° C. 65° C. to 75° C., 70° C. to 90° C., 70° C. to 80° C., 65° C. to 70° C., 70° C. to 75° C., or 75° C. to 95° C. In some embodiments, the low-salt RNA composition is heated for less than 2 minutes, less than 90 seconds, less than 1 minute, less than 50 seconds, less than 40 seconds, less than 30 seconds, less than 20 seconds, or less than 10 seconds. In some embodiments, the low-salt RNA composition is heated for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, the high-salt RNA composition is heated in the presence of a denaturant molecule (e.g., a chemical small molecule that destabilizes or denatures RNA). A denaturant molecule may include dimethyl sulfoxide (e.g., at a concentration of 0.05-1% v/v, 0.1-0.5% v/v, 0.05-0.5% v/v, or 0.25-0.75% v/v), guanidine (e.g., at a concentration of 50-250 mM, 100-500 mM, 250-1000 mM, 1-8 M, 2-6 M, 3-5 M, or 5-8 M), or urea (e.g., at a concentration of 50-250 mM, 100-500 mM, 250-1000 mM, 1-8 M, 2-6 M, 3-5 M, or 5-8 M). In some embodiments, the denaturant molecule is dimethyl sulfoxide, guanidine, urea, ethanol, isopropanol, or acetonitrile.

In some embodiments, a change in the relative amount of denatured RNA in an RNA composition during a denaturation process (e.g., heating the low-salt RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.) is determined by hyperchromicity curves (e.g., spectroscopic melting curves). In some embodiments, a change in the relative amount of denatured RNA is determined by measuring the change in secondary structure of the total RNA in a composition (e.g., by determining a change in ultraviolet absorption). In some embodiments, a change in the relative amount of denatured RNA is determined by monitoring the change in secondary structure of the total RNA in a composition (e.g., by determining a change in ultraviolet absorption) before and after the denaturation process (e.g., heating the low-salt RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.).

In some embodiments, at least 50%, 55%, 60%, 65%, 70%, 75%, 80%, or 85% of the total RNA in a denatured RNA composition comprises denatured RNA. In some embodiments, at least 90% (e.g., at least 91%, 92%, 93%, 94%, 95%, 96%, 97%, 98%, 99% or 100%) of the total RNA in a denatured RNA composition comprises denatured RNA. In some embodiments, 50-70%, 45-60%, 55-70%, 60-80%, 60-100%, 75-100%, 50-95%, 75-95%, 80-100%, 80-90%, 90-95%, 95-100%, 90-99%, or 95-99% of the total RNA in a denatured RNA composition comprises denatured RNA. In some embodiments, the relative amount of denatured RNA in a denatured RNA composition is determined by hyperchromicity curves (e.g., spectroscopic melting curves). Hyperchromicity, the property of nucleic acids such as RNA to exhibit an increase in extinction coefficient upon the loss of structure during heating, may be measured (e.g., during denaturation of RNA, e.g., by heating) using a spectrophotometer. In some embodiments, the extinction coefficient of RNA is measured at 205 nm, 220 nm, 260 nm, or 200-300 nm. In some embodiments, the relative amount of denatured RNA in a denatured RNA composition is determined using a method as described in S. J. Schroeder and D. H. Turner, “Optical melting measurements of nucleic acid thermodynamics”, Methods Enzymol. 468 (2009) 371-387; or Gruenwedel, D. W., “Nucleic Acids: Properties and Determination”, Encyclopedia of Food Sciences and Nutrition, 2003, Pages 4147-4152.

In some embodiments, a denatured RNA composition is stored in a break tank (i.e., a storage container that can hold the denatured RNA composition) prior to mixing with a high-salt buffer and/or loading onto an oligo-dT resin. In some embodiments, the break tank is capable of storing the denatured RNA composition such that the RNA remains denatured for up to 3 days. In some embodiments, the denatured RNA composition is maintained at a low salt concentration (e.g., 1-20 mM, 1-15 mM, 1-10 mM, 1-5 mM, 5-20 mM, 5-10 mM, 10-20 mM, 10-15 mM or 15-20 mM salt) in the break tank. In some embodiments, the break tank is capable of storing the denatured RNA composition such that the RNA remains denatured for 1-6 hours, 2-12 hours, 5-15 hours, 12-24 hours, 12-36 hours, 1-2 days, 1-3 days, or 2-3 days. In some embodiments, the break tank is maintained at 15° C. to 30° C., 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

### In-Line Mixing

In some embodiments, in-line mixing refers to mixing of a first continuous stream of a solution with a second continuous stream of a solution. In some embodiments, the first and second continuous streams are controlled by independent pumps (e.g., independent peristaltic pumps). In some embodiments, in-line mixing relies on flow control conditions, for example, process flow conditions wherein flow parameters (e.g., flow rate, temperature) are controlled by a flow regulating device comprising at least one pump system. In some embodiments, the first continuous stream is a high-salt buffer (e.g., comprising at least 500 mM salt), and the second continuous stream is a composition comprising desalted (e.g., low-salt) and/or denatured RNA.

In-line mixing typically occurs shortly prior to binding a composition comprising denatured RNA to an oligo-dT resin. In some embodiments, in-line mixing occurs for less than 2 minutes, less than 90 seconds, less than 1 minute, less than 50 seconds, less than 40 seconds, less than 30 seconds, less than 20 seconds, or less than 10 seconds. In some embodiments, in-line mixing occurs for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, in-line mixing occurs 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds prior to binding a composition comprising denatured RNA to an oligo-dT resin. In some embodiments, in-line mixing occurs at a temperature of 4° C. to 30° C., 4° C. to 25° C., 4° C. to 20° C. 4° C. to 15° C., 4° C. to 10° C., 4° C. to 8° C. 10° C. to 30° C. 10° C. to 25° C. 10° C. to 20° C. 10° C. to 15° C., or 15° C. to 25° C.

In some embodiments, a high-salt buffer (e.g., that may be in-line mixed with an RNA composition) comprises a salt concentration of at least 500 mM, at least 600 mM, at least 700 mM, at least 800 mM, at least 900 mM, or at least 1000 mM. In some embodiments, a high-salt buffer comprises a salt concentration of 500-1000 mM, 500-600 mM, 500-700 mM, 500-750 mM. 700-1000 mM, 750-900 mM, or 850-1000 mM. In some embodiments, a high-salt buffer comprises a salt concentration of 1-2 M. 2-3 M. 3-4 M, or 4-5 M. In some embodiments, a high-salt buffer comprises a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, a high-salt buffer comprises a conductivity of 5-10 mS/cm, 5-15 mS/cm, 5-7 mS/cm. 6-9 mS/cm, 8-10 mS/cm, 9-12 mS/cm, or 10-15 mS/cm.

In some embodiments, in-line mixing a composition comprising denatured RNA with a high-salt buffer produces a composition comprising denatured RNA and salt at a concentration of at least 500 mM. In some embodiments, in-line mixing a composition comprising denatured RNA with a high-salt buffer produces a composition comprising denatured RNA and salt at a concentration of 500-1000 mM, 500-600 mM, 500-700 mM, 500-750 mM, 700-1000 mM. 750-900 mM, or 850-1000 mM. In some embodiments, in-line mixing a composition comprising denatured RNA with a high-salt buffer produces a composition comprising denatured RNA and salt having a conductivity of less than 2 mS/cm. In some embodiments, in-line mixing a composition comprising denatured RNA with a high-salt buffer produces a composition comprising denatured RNA and salt having a conductivity of 2-5 mS/cm, 2-7 mS/cm, 5-10 mS/cm, 5-15 mS/cm, 5-7 mS/cm, 6-9 mS/cm, 8-10 mS/cm, 9-12 mS/cm, or 10-15 mS/cm.

In some embodiments, a buffer (e.g., a low-salt or high-salt buffer) comprises NaCl, KCl, LiCl, NaH₂PO₄, Na₂HPO₄, or Na₃PO₄. In some embodiments, a buffer (e.g., a low-salt or high-salt buffer) comprises any source of sodium, potassium, magnesium, phosphate, chloride, or any other source of salt ions. In some embodiments, a buffer (e.g., a low-salt or high-salt buffer) may further comprise a buffering agent in order to maintain a consistent pH. In some embodiments, a buffer (e.g., a low-salt or high-salt buffer) comprises a neutral pH. In some embodiments, a buffer (e.g., a low-salt or high-salt buffer) comprises a pH of about 6, about 6.5, about 7, about 7.4, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis [2-hydroxyethyl]amino)-2-hydroxypropanesulfonic acid (DIPSO), N-(2-hydroxyethyl) piperazine-N′-(2-hydroxypropanesulfonic acid) (HEPPSO), 4-(2-hydroxyethyl)-1-piperazine propanesulfonic acid (EPPS), N-[tris (hydroxymethyl)-methyl]glycine (Tricine), N,N-bis(2-hydroxyethyl) glycine (Bicine), [(2-hydroxy-1,1-bis (hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris (hydroxymethyl) aminomethane (Tris), and bis [2-hydroxyethyl]iminotris-[hydroxymethyl] methane (Bis-Tris). Other buffers compositions, buffer concentrations, and additional components of a solution for use herein will be apparent to those skilled in the art.

In some embodiments, in-line mixing comprises in-line cooling of a composition comprising denatured RNA to a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C. In some embodiments, in-line mixing comprises in-line cooling of a composition comprising denatured RNA to a temperature below 60° C., 55° C., 50° C., 45° C., 40° C., 35° C., 30° C., 25° C., 20° C., 15° C., 10° C., or 5° C. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at a total salt concentration of less than 20 mM, less than 15 mM, less than 10 mM, less than 5 mM, or less than 1 mM. In some embodiments, during in-line cooling, a composition comprising denatured RNA is maintained at a total salt concentration of 1-20 mM, 1-15 mM, 1-10 mM, 1-5 mM, 5-20 mM, 5-10 mM, 10-20 mM, 10-15 mM or 15-20 mM. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at less than 2.5 mS/cm, less than 2 mS/cm, less than 1.5 mS/cm, less than 1 mS/cm, or less than 0.5 mS/cm. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

### Oligo-dT Resin

The methods herein involve binding (i.e., contacting) compositions comprising denatured RNA to oligo-dT resin. In some embodiments, methods herein comprise binding compositions comprising denatured RNA to oligo-dT resin following in-line mixing of low-salt denatured RNA composition with high-salt buffers.

The methods described herein may use any oligo-dT resin. In some embodiments, the oligo-dT resin is a (poly) styrene-divinylbenzene (PS-DVB) bead resin with 2000 Angstrom pores derivatized with poly dT. In some embodiments, poly dT comprises 5-200, 10-50, 10-100, 50-200, 100-150, or 125-200 thymidines and/or uracils. In some embodiments, poly dT comprises 20 thymidines in length. In some embodiments, poly dT is linked directly to the bead resin. In some embodiments, poly dT is linked to the bead resin via a linker.

In some embodiments, the oligo-dT resin is equilibrated with a buffer prior to binding the RNA to the resin. In some embodiments, the oligo-dT resin is equilibrated with a buffer comprising 100 mM NaCl, 10 mM Tris, and 1 mM EDTA at pH 7.4. In some embodiments, the oligo-dT resin is washed with a buffer after the RNA is bound to the resin. In some embodiments, the washing step comprises a buffer comprising 100 mM NaCl, 10 mM Tris, and 1 mM EDTA, at pH 7.4.

In some embodiments, the methods described herein involve adding a high-salt buffer to a mixture comprising mRNA in order to increase the salt concentration prior to addition of the mixture to an oligo-dT resin. Addition of a high-salt buffer promotes formation of compact secondary structures by mRNAs, while leaving the poly(A) tail exposed. Exposure of the poly (A) tail allows mRNAs to bind to stationary phases such as oligo-dT resin, but the compact structure induced by high salt concentrations reduces steric interactions in which a portion of a stationary phase-bound first mRNA prevents a second mRNA from binding to the stationary phase. Thus, adding a high-salt mRNA composition to the resin causes more RNA molecules to bind to the stationary phase than addition of an equivalent mRNA composition with a lower salt concentration. However, mRNA must be dissolved in a solution in order to pass through a stationary phase. The benefits of high salt concentrations for increasing binding of mRNA to stationary phase must therefore be balanced with the risk of mRNA precipitation. Surprisingly, precipitation requires an extended amount of time even at high salt concentrations. Accordingly, addition of a high-salt buffer to an mRNA composition to produce a high-salt mRNA composition followed by adding the high-salt mRNA composition to a stationary phase shortly thereafter, allows the benefits of high salt concentrations for stationary phase binding to be realized without a consequent reduction in yield due to mRNA precipitation.

In some embodiments, the high-salt buffer is added to the RNA composition by in-line mixing. In some embodiments, in-line mixing refers to mixing of a first continuous stream of a solution with a second continuous stream of a solution. In some embodiments, the first and second continuous streams are controlled by independent pumps (e.g., independent peristaltic pumps). In some embodiments, in-line mixing relies on flow control conditions, for example, process flow conditions wherein flow parameters (e.g., flow rate, temperature) are controlled by a flow regulating device comprising at least one pump system. In some embodiments, the first continuous stream is a high-salt buffer (e.g., comprising at least 500 mM salt), and the second continuous stream is a composition comprising RNA. In some embodiments, the RNA composition has been desalted (e.g., is a low-salt RNA composition) and/or comprises denatured RNA. In-line mixing typically occurs shortly prior to binding a composition comprising RNA to a stationary phase. In some embodiments, in-line mixing occurs for less than 2 minutes, less than 90 seconds, less than 1 minute, less than 50 seconds, less than 40 seconds, less than 30 seconds, less than 20 seconds, or less than 10 seconds. In some embodiments, in-line mixing occurs for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, in-line mixing occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C. 10° C. to 20° C., 10° C. to 15° C., or 15° C. to 25° C.

In some embodiments, the high-salt buffer is added to the RNA composition by bolus addition. In contrast to in-line mixing in which two liquid streams are combined continuously, bolus addition involves the discrete addition of one composition to another. Bolus addition may occur over several seconds or minutes, and be followed by mixing to incorporate the high-salt buffer throughout the RNA composition, thereby distributing salts of the high-salt buffer throughout the RNA composition.

In some embodiments, the high-salt buffer is added to the RNA composition 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds prior to binding a composition comprising RNA to a stationary phase. In some embodiments, adding the high-salt buffer occurs 1-60 minutes, 1-45 minutes, 1-30 minutes, 1-25 minutes, 1-20 minutes, 1-15 minutes, 1-10 minutes, 1-5 minutes, or 1-2 minutes prior to adding the high-salt RNA composition to the stationary phase. In some embodiments, adding the high-salt RNA composition to the stationary phase occurs within 1 hour or less, 45 minutes or less, 30 minutes or less, 25 minutes or less, 20 minutes or less, 15 minutes or less, 10 minutes or less, 9 minutes or less, 8 minutes or less, 7 minutes or less, 6 minutes or less, 5 minutes or less, 4 minutes or less, 3 minutes or less, 2 minutes or less, or 1 minute or less of adding the high-salt buffer to the RNA composition. In some embodiments, the high-salt buffer, the RNA composition, and/or the high-salt RNA composition produced by adding the high-salt buffer has a temperature of 4° C. to 30° C. 4° C. to 25° C., 4° C. to 20° C. 4° C. to 15° C., 4° C. to 10° C., 4° C. to 8° C., 10° C. to 30° C. 10° C. to 25° C. 10° C. to 20° C., 10° C. to 15° C. or 15° C. to 25° C.

In some embodiments, a high-salt buffer (e.g., that may be mixed with an RNA composition) comprises a salt concentration of at least 500 mM, at least 600 mM, at least 700 mM, at least 800 mM, at least 900 mM, or at least 1000 mM. In some embodiments, a high-salt buffer comprises a salt concentration of 500-1000 mM, 500-600 mM, 500-700 mM, 500-750 mM. 700-1000 mM, 750-900 mM, or 850-1000 mM. In some embodiments, a high-salt buffer comprises a salt concentration of 1-2 M. 2-3 M, 3-4 M, or 4-5 M. In some embodiments, a high-salt buffer comprises a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, a high-salt buffer comprises a conductivity of 5-10 mS/cm, 5-15 mS/cm, 5-7 mS/cm. 6-9 mS/cm, 8-10 mS/cm, 9-12 mS/cm, or 10-15 mS/cm.

In some embodiments, mixing a composition comprising RNA with a high-salt buffer produces a high-salt RNA composition comprising RNA and salt at a concentration of at least 100 mM. In some embodiments, mixing a composition comprising RNA with a high-salt buffer produces a composition comprising RNA and salt at a concentration of 500-1000 mM, 500-600 mM, 500-700 mM, 500-750 mM, 700-1000 mM. 750-900 mM, or 850-1000 mM. In some embodiments, the salt concentration of the high-salt RNA composition is at least 500 mM, at least 600 mM, at least 700 mM, at least 800 mM, at least 900 mM, at least 1 M, or more. In some embodiments, the high-salt RNA composition has a salt concentration of about 500 mM to about 600 mM, about 600 mM to about 800 mM, about 800 mM to about 1 M, about 1 M to about 1.5 M, about 1.5 M to about 2 M, about 2 M to about 2.5 M, about 2.5 M to about 3 M, about 3 M to about 4 M, or about 4 M to about 5 M. In some embodiments, the high-salt mRNA composition has a salt concentration of about 500 mM to about 600 mM. In some embodiments, the high-salt RNA composition comprises a salt concentration of about 500 mM. In some embodiments, the salt concentration of the high-salt RNA composition refers to the concentration of sodium chloride, potassium chloride, ammonium chloride, ammonium sulfate, monosodium phosphate, disodium phosphate, or trisodium phosphate in the composition. In some embodiments, mixing a composition comprising RNA with a high-salt buffer produces a composition comprising RNA and salt having a conductivity of less than 2 mS/cm. In some embodiments, mixing a composition comprising RNA with a high-salt buffer produces a composition comprising RNA and salt having a conductivity of 2-5 mS/cm, 2-7 mS/cm, 5-10 mS/cm, 5-15 mS/cm, 5-7 mS/cm, 6-9 mS/cm, 8-10 mS/cm, 9-12 mS/cm, or 10-15 mS/cm.

In some embodiments, a buffer (e.g., a low-salt or high-salt buffer) comprises NaCl, KCl, LiCl, NaH₂PO₄, Na₂HPO₄, or Na₃PO₄. In some embodiments, a buffer (e.g., a low-salt or high-salt buffer) comprises any source of sodium, potassium, magnesium, phosphate, chloride, or any other source of salt ions. In some embodiments, a buffer (e.g., a low-salt or high-salt buffer) may further comprise a buffering agent in order to maintain a consistent pH. In some embodiments, a buffer (e.g., a low-salt or high-salt buffer) comprises a neutral pH. In some embodiments, a buffer (e.g., a low-salt or high-salt buffer) comprises a pH of about 6, about 6.5, about 7, about 7.4, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis [2-hydroxyethyl]amino)-2-hydroxypropanesulfonic acid (DIPSO), N-(2-hydroxyethyl) piperazine-N′-(2-hydroxypropanesulfonic acid) (HEPPSO), 4-(2-hydroxyethyl)-1-piperazine propanesulfonic acid (EPPS), N-[tris (hydroxymethyl)-methyl]glycine (Tricine), N,N-bis(2-hydroxyethyl) glycine (Bicine), [(2-hydroxy-1,1-bis (hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris (hydroxymethyl) aminomethane (Tris), and bis [2-hydroxyethyl]iminotris-[hydroxymethyl] methane (Bis-Tris). Other buffers compositions, buffer concentrations, and additional components of a solution for use herein will be apparent to those skilled in the art.

In some embodiments, the binding of the RNA to the oligo-dT resin occurs at a temperature of lower than 40° C. In some embodiments, the binding of the RNA to the oligo-dT resin occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the composition comprising denatured RNA is bound to or in contact with the oligo-dT resin for a total residence time of less than 20 minutes, less than 18 minutes, less than 15 minutes, less than 12 minutes, less than 10 minutes, less than 9 minutes, less than 8 minutes, less than 7 minutes, less than 6 minutes, less than 5 minutes, less than 4 minutes, less than 3 minutes, less than 2 minutes, or less than 1 minute. In some embodiments, the composition comprising denatured RNA is bound to or in contact with the oligo-dT resin for a total residence time of 1-2, 1-5, 2-5, 2-10, 5-20, 5-10, 5-15, 8-15, 10-15, 12-20, or 15-20 minutes.

In some embodiments, the methods comprise eluting RNA from the oligo-dT resin. In some embodiments, the RNA is eluted from the HIC resin using water or a buffer (e.g., a buffer comprising 10 mM Tris, 1 mM EDTA, at pH 8.0).

In some embodiments, the secondary structure of the RNA is monitored using ultraviolet detection before and after applying the RNA to the oligo-dT resin. In some embodiments, monitoring of RNA using UV detection can be performed using an in-line detector. In some embodiments, monitoring of RNA involves taking a measurement of a sample of RNA before applying the RNA to the oligo-dT resin and then a second measurement of a second sample of RNA after the RNA has been applied to the oligo-dT resin.

In some embodiments, denatured RNA is refolded (e.g., naturally refolded) following elution from the oligo-dT resin. In some embodiments, RNA is applied to a tangential flow filtration step following elution from the oligo-dT resin.

In some embodiments, the RNA eluted from the oligo-dT resin comprises at least 50%, 55%, 60%, 65%, 70%, 75%, 80%, or 85% poly-A tailed RNA. In some embodiments, the RNA eluted from the oligo-dT resin comprises about 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99% poly-A tailed mRNA. In some embodiments, the RNA eluted from the oligo-dT resin comprises at least 91%, 92%, 93%, 94%, 95%, 96%, 97%, 98%, 99% or 100% poly-A tailed mRNA.

In some embodiments, a method of the disclosure comprises: (a) applying denaturing conditions to a mixture comprising ribonucleic acid (RNA) to produce a composition comprising denatured RNA; (b) binding the denatured RNA to an oligo-dT resin; (c) eluting RNA from the oligo-dT resin; (d) applying the eluted RNA of step (c) to a flow-through column comprising hydrophobic interaction chromatography (HIC) resin; and (e) isolating the RNA of step (d). In some embodiments, denaturing conditions comprise heating the mixture to a temperature of higher than 60° C. and/or incubating the mixture in the presence of a denaturant molecule. In some embodiments, prior to step (a), the mixture is desalted to produce a low-salt mixture. In some embodiments, a low-salt buffer is added to the eluted RNA of step (c) in order to produce a low-salt mixture comprising the eluted RNA. The low-salt mixture comprising the eluted RNA may, in such an embodiment, then be added to the flow-through column comprising HIC resin. The HIC resin may be a high hydrophobicity resin (e.g., Poros R150 resin).

### Apparatus

Some aspects of the present disclosure provide an apparatus for purifying RNA using denaturing dT chromatography. In some embodiments, the apparatus comprises a column packed with oligo-dT resin, the column having an inlet and an outlet. In some embodiments, the apparatus is a flow regulating device comprising at least one pump system, wherein the pump system allows for continuous blend mixing of two or more solutions under flow control conditions (e.g., process flow conditions) to control flow parameters such as flow rate and temperature of the two or more solutions to be mixed. In some embodiments, the apparatus comprises, upstream of the inlet of the column, a first continuous stream for delivering desalted RNA, wherein the flow of desalted RNA is controlled by a first pump, and wherein the first stream in encased within a denaturation chamber comprising a pre-heater followed by a chiller; a second stream for delivering high-salt buffer, wherein the flow of high-salt buffer is controlled by a second pump; and a chamber where the two continuous streams are combined to provide in-line mixing of the desalted RNA and high-salt buffer.

In some embodiments, the apparatus comprises an oligo-dT resin that comprises (poly) styrene-divinylbenzene (PS-DVB) bead resin with 2000 Angstrom pores derivatized with poly dT.

In some embodiments, a column (e.g., a column packed with oligo-dT resin) has a column volume of 1-10 mL, 1-5 mL, 5-25 mL, 10-100 mL, 25-150 mL, 50-100 mL, 75-150 mL, 100-200 mL, 100-500 mL, 250-1000 mL, 500-1500 mL, or more. In some embodiments, a column (e.g., a column packed with oligo-dT resin) has a column volume of about 1 mL, about 5 mL, about 10 mL, about 25 mL, about 50 mL, about 100 mL, about 250 mL, about 500 mL, about 750 mL, about 1000 mL, about 1500 mL, about 2000 mL, or more.

In some embodiments, the pre-heater heats the desalted RNA to a temperature of higher than 60° C. to produce a denatured RNA composition. In some embodiments, the pre-heater is maintained at 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.

In some embodiments, the chiller cools the denatured RNA composition to a temperature of less than 30° C. In some embodiments, the chiller is maintained at 15° C. to 30° C., 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the apparatus further comprises a break tank (i.e., a storage container that can hold the denatured RNA composition). In some embodiments, the break tank is capable of storing the denatured RNA composition such that the RNA remains denatured for up to 3 days. In some embodiments, the denatured RNA composition is maintained at a low salt concentration (e.g., 1-20 mM, 1-15 mM, 1-10 mM, 1-5 mM, 5-20 mM, 5-10 mM, 10-20 mM, 10-15 mM or 15-20 mM salt) in the break tank. In some embodiments, the break tank is capable of storing the denatured RNA composition such that the RNA remains denatured for 1-6 hours, 2-12 hours, 5-15 hours, 12-24 hours, 12-36 hours, 1-2 days, 1-3 days, or 2-3 days. In some embodiments, the break tank is maintained at 15° C. to 30° C. 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the apparatus further comprises at least one ultraviolet detection (UV) module. In some embodiments, the UV detection module is positioned to detect RNA using UV light during the denaturation process (e.g., during a heating step intended to denature the RNA). In some embodiments, the UV detection module is positioned to detect RNA using UV light after the denaturation process (e.g., after a heating step intended to denature the RNA). In some embodiments, the apparatus comprises two UV modules. In some embodiments, the apparatus comprises a first UV module positioned to detect RNA using UV light before the denaturation process (e.g., before a heating step intended to denature the RNA) and a second UV module positioned to detect RNA using UV light after the denaturation process (e.g., after a heating step intended to denature the RNA).

In some embodiments, the apparatus is used to process desalted RNA produced using an in vitro transcription reaction. In some embodiments, the high-salt buffer has a salt concentration of at least 500 mM. In some embodiments, the high-salt buffer has a salt concentration of 500 mM to 1000 mM NaCl.

### EXAMPLES

### Example 1. Evaluation of HIC Resins

The ability of various HIC resins to discriminate between RNA and proteins (e.g., in order to purify RNA from residual proteins following an IVT reaction) was evaluated. Phenyl Sepharose, Capto Butyl ImpRes, Capto Phenyl (High Sub), Poros Benzyl, Poros Benzyl Ultra and Poros R150 were identified as commercially available resins to be tested, each of which was obtained from Cytiva or Thermo Fisher. It was determined that the rank order of hydrophobicity of these resins was Phenyl Sepharose, Capto Butyl ImpRes, Capto Phenyl (High Sub), Poros Benzyl, Poros Benzyl Ultra and Poros R150, with Phenyl Sepharose being the least hydrophobic of the grouping and Poros R150 being the most hydrophobic of the grouping. The resins were tested using a bind-elute mode and a flow-through mode.

The results of the bind-elute mode showed that most of the resins required high salt for binding, with NaCl salt concentrations ranging from 1.5M to 2M. The Poros R150 resin however was capable of binding to residual protein and/or RNA at 500 mM NaCl. Further, for each of the resins, RNA did not elute in 0 mM salt conditions; but instead eluted in 0.5 M NaOH. RNA was also shown to be unstable at high salt concentrations, with precipitation of the RNA occurring at salt conditions of more than 500 mM.

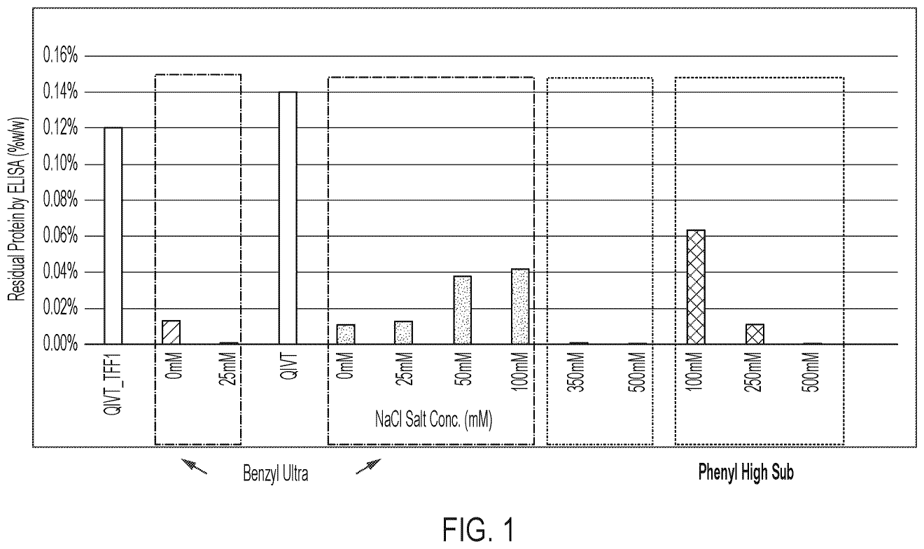

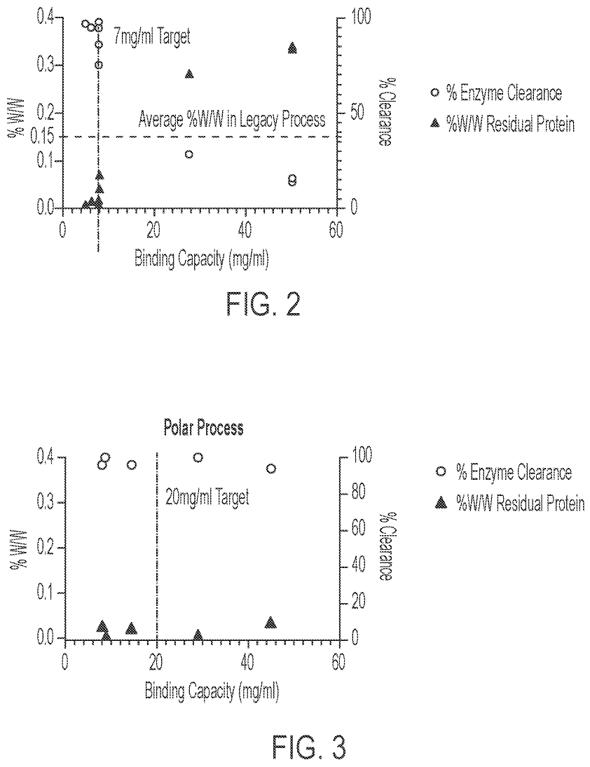

For the flow-through mode testing, columns comprising HIC resin were equilibrated with a buffer solution comprising about 200 mM salt concentration and loaded with samples comprising 3 mg/mL RNA from an IVT reaction. Results from the flow-through mode testing showed that RNA flowed through the column comprising HIC resin without binding to the resin, while proteins bound to the HIC resin. Residual protein (% w/w) following collection of the RNA (e.g., RNA that is free from enzymes) from the flow-through columns, as determined using an ELISA assay, for each of the HIC resins (Poros Benzyl Ultra, Poros Benzyl, and Capto Phenyl (High Sub)) are provided in FIG. 1 and Table 1. Poros Benzyl Ultra resin provided the highest percent recovery and protein clearance when using a 25 mM NaCl washing condition (residual protein not detected using ELISA assay, less than 0.001% residual protein).

TABLE 1
Results from flow-through mode

| | NaCl | RNA Recovery | Residual protein |
|---|---|---|---|
| Resin Type | concentration | (%) | % (w/w) |
| Benzyl Ultra | 0 mM | 111 | 0.013% |
| Benzyl Ultra | 25 mM | 83 | 0.001% |
| Benzyl Ultra | 0 mM | 64 | 0.011% |
| Benzyl Ultra | 25 mM | 57 | 0.013% |
| Benzyl Ultra | 50 mM | 60 | 0.038% |
| Benzyl Ultra | 100 mM | 62 | 0.042% |
| Benzyl | 350 mM | 71 | 0.001% |
| Benzyl | 500 mM | 77 | 0.001% |
| Phenyl High Sub | 100 mM | 77 | 0.063% |
| Phenyl High Sub | 250 mM | 71 | 0.011% |
| Phenyl High Sub | 500 mM | 76 | 0.001% |

Poros Benzyl Ultra was used for further HIC resin testing. Three factors of the resin were tested using a sample comprising RNA produced by an IVT reaction. The first factor tested was load challenge, which varied from 2 to 8 mg/mL RNA. The second factor tested was binding resistance time, which varied from 1.2 to 10 minutes. The third factor tested was salt concentration, which varied from 2 to 20 mM. The experiments demonstrated that the resin could reproducibly allow for high RNA recovery while limiting the residual protein below the limit of detection (e.g., less than 0.001% w/w protein) when using loads of up to 8 mg/mL RNA, allowing for binding times of as low as 1.2 minutes, and using salt concentrations of greater than 2 mM during the load.

### Example 2: Exemplary Process of the Disclosure

An exemplary process of the disclosure was designed and tested for performance in purification of a sample comprising RNA produced by an IVT reaction, relative to a previous method that utilized ambient oligo-dT resin for removal of residual protein. The exemplary process utilized flow-through hydrophobic interaction chromatography (column comprising Poros Benzyl Ultra resin) as the final purification step for isolation of mRNA produced from an IVT reaction from a low-salt mixture comprising RNA and residual protein. A sample comprising RNA produced by an IVT reaction was loaded onto the HIC resin and washed with a low-salt wash buffer (e.g., a buffer comprising 10 mM Tris, 1 mM EDTA, 100 mM NaCl, pH 7.4) followed by a high-salt wash buffer (e.g., a buffer comprising 10 mM Tris, 1 mM EDTA, 500 mM NaCl, pH 7.4). The RNA was eluted from the resin using an elution buffer (e.g., a buffer comprising 10 mM Tris, 1 mM EDTA, pH 7.4). The exemplary process further comprised a denaturing oligo-dT performed under low-salt conditions and subsequent high salt adjustment prior to the flow-through HIC step.

The previous method could be used to purify RNA from an IVT reaction at a load of 2 g/L with a total processing time of 24 hours. Comparatively, the exemplary, novel method could be used to purify RNA from an IVT reaction at a load of 8 g/L with a total processing time of only two (2) hours. Liquid chromatography/mass spectrometry (LC/MS) was also used to determine the amount of residual protein following discrete steps of the exemplary process

(Table 2). In contrast to samples purified using known methods such as oligo-dT, the methods disclosed herein result in significant reductions in residual protein.

TABLE 2
Residual protein following discrete steps of the exemplary process

| | | | T7 | Total | Residual |
|---|---|---|---|---|---|
| | DNAseI | Ppiase | polymerase | Protein | Protein |
| Step | (μg/mL) | (μg/mL) | (μg/mL) | (μg/ml) | % w/w |
| Tangential flow | <LOQ | 0.3436105 | 2.94 | 3.28 | 0.33 |
| filtration prior | | | | | |
| to oligo-dT | | | | | |
| denaturing | <LOQ | <LOQ | 1.29 | 1.29 | 0.13 |
| oligo-dT | | | | | |
| flow-through | <LOQ | <LOQ | 0.16 | 0.16 | 0.02 |
| HIC | | | | | |

The exemplary method was replicated using an additional sample comprising RNA produced by a discrete IVT reaction. At the flow-through HIC step, the column comprising HIC resin was loaded with 8 mg/mL RNA over a 5-minute residence time at 200 mM NaCl. RNA recovery was measured by UV spectroscopy quantification. Liquid chromatography/mass spectrometry (LC/MS) was used to determine the amount of residual protein and concentration of mRNA following discrete steps of the exemplary process (Table 3).

TABLE 3
Residual protein following discrete steps of the exemplary process

| | Total | mRNA | Residual |
|---|---|---|---|
| | Protein | concentration | Protein |
| Step | (μg/ml) | (mg/ml) | % w/w |
| Tangential flow filtration | 4.777 | 1.03 | 0.465 |
| prior to oligo-dT | | | |
| denaturing oligo-dT | 2.122 | 1.02 | 0.209 |
| flow-through HIC | 0.053 | 0.81 | 0.006 |

The exemplary method was shown to be a more efficient process for protein clearance while maintaining RNA (and thus producing highly pure samples of RNA), with greater than 94% protein clearance at a throughput of up to 15 times greater. Additional differences between the exemplary method and the previous method are provided in Table 4.

TABLE 4

| | | Exemplary method | |
|---|---|---|---|
| | Previous | using denaturing | Fold |
| | method | oligo-dT and FT HIC | Improvement |
| Binding Capacity | 2.0 | 20.0 | 10.0 |
| Target (g/L) | | | |
| Process Time per | 95.0 | 60.0 | 0.6 |
| Cycle (min) | | | |
| Column Volume (L) | 1.0 | | |
| Cycles | 6.0 | 1.0 | 0.2 |
| Processing Time (hrs) | 9.5 | 1.0 | 0.1 |
| Chromatography | 1.3 | 20.0 | 15.8 |
| Productivity (g/L/h) | | | |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A method comprising:
applying a mixture comprising messenger ribonucleic acid (mRNA) produced by an in vitro transcription reaction to a flow-through column comprising hydrophobic interaction chromatography (HIC) resin, wherein the mixture comprising mRNA is a low-salt mixture, and wherein the HIC resin has high hydrophobicity.

2. A method comprising:
applying a mixture comprising messenger ribonucleic acid (mRNA) produced by an in vitro transcription reaction and residual protein to a flow-through column comprising hydrophobic interaction chromatography (HIC) resin under conditions that do not allow for the mRNA to substantially bind to the HIC resin but do allow for the residual protein to bind to the HIC resin.

3. The method of claim 2, wherein conditions that do not allow for the mRNA to substantially bind to the HIC resin but do allow for the residual protein to bind to the HIC resin comprise low-salt conditions.

4. The method of claim 1, further comprising desalting the mixture prior to applying the mixture to the flow-through column.

5. The method of claim 1, wherein the low-salt mixture comprises a salt concentration of less than 20 mM.

6. The method of claim 1, wherein the low-salt mixture comprises a salt concentration of 0-500 mM.

7. The method of claim 1, wherein the HIC resin is equilibrated prior to the applying step using a buffer solution comprising 0-100 mM salt concentration.

8. The method of claim 1, wherein, following the applying step, the HIC resin is washed with a buffer solution comprising 0-100 mM salt concentration.

9. The method of claim 5, wherein the salt comprises an alkali metal cation.

10. The method of claim 5, wherein the salt comprises an alkaline earth metal.

11. The method of claim 9, wherein the mixture or buffer solution further comprises a counterion.

12. The method of claim 5, wherein the salt comprises an anti-chaotropic salt.

13. The method of claim 1, wherein the mRNA does not substantially bind to the hydrophobic interaction resin and/or residual protein binds to the HIC resin.

14. The method of claim 1, wherein the HIC resin comprises a cross-linked poly (styrene-divinylbenzene) matrix with an aromatic hydrophobic benzyl ligand and an average particle size of 50 μm.

15. The method of claim 1, wherein the HIC resin comprises a hydrophobic moiety selected from butyl, t-butyl, phenyl, ether, amide, or propyl groups.

16. The method of claim 1, wherein the mRNA being applied to the HIC resin comprises at least 90% poly-A tailed mRNA.

17. The method of claim 1, further comprising isolating the mRNA from the flow-through column.

18. The method of claim 17, further comprising applying the RNA to a tangential flow filtration step following isolating the mRNA from the flow-through column.

19. The method of claim 3, wherein the mixture comprising mRNA is a low-salt mixture.

20. The method of claim 6, wherein the low-salt mixture comprises a salt concentration of 0-350 mM.
